(19) United States
(12) Patent Application Publication
(10) Pub. No.: US 2015/0190534 A1
(43) Pub. Date: Jul. 9, 2015

(54) COMPOUNDS FOR USE AS POSITRON EMISSION IMAGING AGENTS

(71) Applicant: UNIVERSITY OF BERN, Bern (CH)

(72) Inventors: Brigitte FREY, Bolligen (CH); Reto BERTOLLINI, Liebefeld (CH)

(73) Assignee: UNIVERSITY OF BERN, Bern (CH)

(21) Appl. No.: 14/151,501

(22) Filed: Jan. 9, 2014

(51) Int. Cl. A61K 51/04; C07J 1/00

(52) U.S. Cl. CPC A61K 51/0493; C07J 1/0014

(57) ABSTRACT

The present invention relates generally to compounds according to formula I that bind more specifically to mutated androgen receptors than to the wild type androgen receptors and therefore are useful as imaging agents for positron emission tomography (PET) used in the diagnosis and monitoring of prostate cancer.

### FIELD OF THE INVENTION

The present invention relates generally to compounds according to formula I that bind more specifically to mutated androgen receptors than to the wild type androgen receptors, and therefore are useful as imaging agents for positron emission tomography (PET) used in the diagnosis and monitoring of prostate cancer and its metastases.

### BACKGROUND ART

Globally prostate cancer (PC) is the sixth leading cause of cancer-related death in men. Several treatment strategies are available such as surgery, radiation and hormone therapy without and in combination with chemotherapy. While natural testosterone promotes any prostatic cell and cannot discriminate between receptors of healthy and cancerous tissue, prostate cancer hormone therapy aims to slow or stop growth and spreading of prostate cancer. In this respect bicalutamide was launched 1995 as an oral non-steroidal antiandrogen, binding to androgen receptors (AR) and preventing the activation of the AR and subsequent upregulation of androgen responsive genes. Initially, nearly all PCs are androgen dependent while with time most PC cells relapse and start growing after their initial response. Despite the treatment with anti-androgens, the AR signaling continues, the bioavailability of the ligands increases together with the AR expression; structural changes of AR by mutations occur with changes of AR co-regulators levels. This so-called “antiandrogen withdrawal syndrome” has been observed in 30% to 50% of patients. In 2003 Hara et al. demonstrated that bicalutamide was able to cause the anti-androgen withdrawal phenomenon via AR mutation in PC cells (Hara T, Miyazaki J, Araki H, et al. Novel mutations of androgen receptor: a possible mechanism of bicalutamide withdrawal syndrome. Cancer Res. Jan. 1, 2003; 63(1): 149-153.). Instead of presenting antagonist action, bicalutamide worked as an agonist for W741C-AR and W741L-AR mutant, while flutamide worked as an antagonist for these mutants. Taplin et al. reported that T877A is a hot spot of the AR mutation selected by treatment with flutamide in PC patients (Taplin M E, Bubley G J, Ko Y J, et al. Selection for androgen receptor mutations in prostate cancers treated with androgen antagonist. Cancer Res. Jun. 1, 1999; 59(11):2511-2515.), while Hara et al. observed the phenomenon of flutamide withdrawal syndrome in which tumors regressed after anti-androgen treatment was stopped. This T877A mutation can be antagonized in vitro by bicalutamide and mifepristone (Song L N, Coghlan M, Gelmann E P. Antiandrogen effects of mifepristone on coactivator and corepressor interactions with the androgen receptor. Mol Endocrinol. January 2004; 18(1):70-85).

Recently, it was demonstrated that dihydrotestosterone (DHT) derivatives can efficiently bind and antagonize wild type (WT) but also four different forms of mutant AR (Andrieu T, Bertolini R, Nichols S E, et al. A novel steroidal antiandrogen targeting wild type and mutant androgen receptors. Biochem Pharmacol. Dec. 1, 2011; 82(11):1651-1662.).

To detect potential mutations of the AR in PC and its metastases, biopsies represent the only tool so far, but are nevertheless invasive and hazardous. Furthermore, there are risks of sampling errors, where mutation-negative tissue is biopsied while mutation-positive tissue remains untouched.

Finally, obtaining biopsies of all cancerous lesions is virtually impossible in patients with multiple metastases. The ideal tool for pre-therapeutic detection of the T877A-AR or other mutations would be a non-invasive method preventing any sampling errors while assessing all metastases throughout the body.

In this respect, positron-emission tomography (PET) represents a sensitive tool for noninvasive imaging of all tissues throughout the entire body. Currently, for PET imaging ¹⁸F-fluorodeoxyglucose (¹⁸F-FDG) and ¹⁸F-fluorocholine (¹⁸F-FCH) are injected as radioactive tracers into the blood stream. These tracers are designed to accumulate in cells with specific biochemical properties.

It has been previously shown the feasibility to design a tracer that detects mutations of the enzyme deoxycytidine kinase and thereby predicts the success of gemcitabine therapy even before the start of treatment (Laing R E, Walter M A, Campbell D O, et al. Noninvasive prediction of tumor responses to gemcitabine using positron emission tomography. Proc Natl Acad Sci USA. Feb. 24, 2009; 106(8):2847-2852.).

### SUMMARY OF THE INVENTION

The summary is not intended to limit the scope of the present invention. Additional embodiments will be apparent from the detailed description, drawings and from the claims.

The present invention provides compounds, also referred to herein as ligands, of the following basic, fundamental formula I:

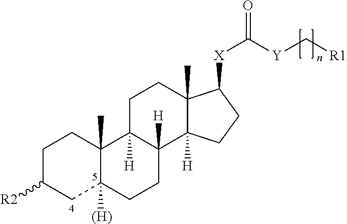

wherein R1 is ¹⁸F, ¹⁹F, OH, or CH₃; R2 is ═O, OH, ¹⁸F, or ¹⁹F; X is O or NH; Y is NH, O, CH₂, or S; n is an integer from 2-12; and 4,5-bond is a single or double bond; and wherein if R1 is CH₃, then n is an integer from 0-11.

More specifically, the present invention provides compounds 1, 2, 3, 4, and 5 of the following formulas 1-5:

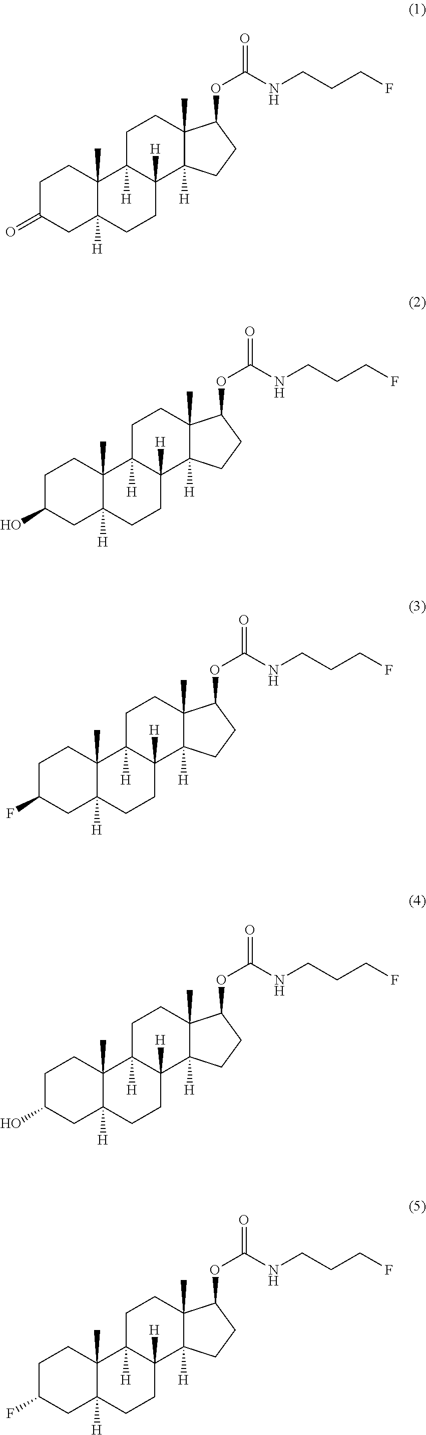

The present invention also provides compound of formula I and compounds of 1, 2, 3, 4, or 5 which bind more specifically to mutated androgen receptors than to the wild type androgen receptor, and therefore are useful as imaging agents for positron emission tomography (PET) used in the diagnosis and monitoring of prostate cancer.

The present invention further relates to radiopharmaceutical compositions comprising compound of formula I or compounds of 1, 2, 3, 4, or 5 together with a biocompatible carrier in a form suitable for administration to a mammal.

Additional embodiments provide methods and use of the compositions according to any of the preceding embodiments in the diagnosis and monitoring of prostate cancer.

### DRAWINGS

Those of skill in the art will understand that the drawings, described below, are for illustrative purposes only. The drawings are not intended to limit the scope of the present teachings in any way.

FIG. 1. Illustrates synthesis of DHT derivatives RB390, ¹⁸F-RB390, 3a-hydroxy-RB448, 3b-hydroxy-RB448, 3a-hydroxy-¹⁸F-RB448 and 3b-hydroxy-¹⁸F-RB448.

FIG. 2. Illustrates synthesis of DHT derivatives 3a-fluoro-RB448, 3b-fluoro-RB448, 3a-¹⁸F-fluoro-RB448 and 3b-¹⁸F-fluoro-RB448.

FIG. 3. Illustrates binding characteristics (IC₅₀) of DHT (open quadrangles), RB390 (closed circle) and 3-hydroxy-RB448 (closed triangle) with respect to WT-AR (A) and T877A-AR (B) determined by whole cell competitive binding assay. COS-7 cells transfected with human WT-AR or T877A-AR were incubated dose-dependently with either ligand and IC₅₀ and relative binding affinity (RBA) determined. RBA for DHT was considered as 100%.

FIG. 4. Illustrates side-by-side comparison of experimental ligands with Induced Fit Docking predictions of RB390. AR co-crystal structures (left) and RB390 docked (right) into 1137 WT (top) and 2OZ7 T877A (bottom). Results of both indicate that the side-chain arm points towards the N-terminal end of helix H12. Helices are hidden in docked renderings for better view of interacting residues.

FIG. 5. Illustrates uptake of radioligands by AR overexpressing cells. COS-7 cells transfected with empty vector (pcDNA3), WT-AR (pSG5AR) or mutant T877A-AR (pSG5AR-T877A) were incubated with ¹⁸F-RB390. New PET ligand for T877A-AR mutant ¹⁸F-RB390 (A, B), ¹⁸F-FCH (C) or ¹⁸F-FDG (D) for 10 min and washed once with ice-cold PBS (A, C, D) or 3× with CT-DMEM containing 2% FBS at a 10 min interval (A, B, C, D). Radioactivity was measured and corrected by the protein content. Specificity was demonstrated by incubating transfected COS-7 cells with ¹⁸F-RB390 in absence or presence (hatched bars) of excessive DHT.

FIG. 6. Illustrates tumor-to-heart ratios of male SCID mice bearing subcutaneous C4-2 (+, with AR) and PC-3 (−, without AR) tumor xenografts 3 h after injection of ¹⁸F-RB390 are presented. C4-2 blocked indicates injection of 3 μg unlabelled DHT in parallel.

FIG. 7. Illustrates PET/CT scan of PC-3 (−, without AR) and C4-2 (+, with AR) tumor bearing mice 3 h after injection of ¹⁸F-RB390. Radioactivity assessed in C4-2 tumors is significant and absent in PC-3 tumors (A, circled areas). The radioactive signal in C4-2 cells however is remarkably reduced to background levels when an excess of unlabelled DHT was injected in parallel (B).

### DETAILED DESCRIPTION OF THE INVENTION

The present invention is described further as follows.

In one embodiment, the present invention relates to compounds of formula I to detect specifically wild type and mutated ARs in PC and its corresponding metastases. The compounds of formula I is:

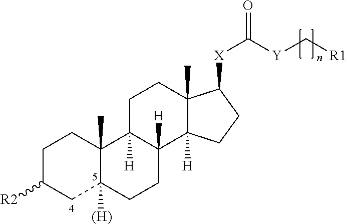

wherein R1 is ¹⁹F, OH, or CH₃; R2 is ═O, OH in α- or β-position, ¹⁸F or ¹⁹F in α- or β-position; X is O or NH; Y is NH, O, CH₂, or S; n is an integer from 2-12; and 4,5-bond is a single or double bond; and wherein if R1 is CH₃, then n is an integer from 0-11.

Optionally, when R1 is ¹⁸F or ¹⁹F, R2 is ═O. Optionally, when R1 is ¹⁸F or ¹⁹F, R2 is OH in α- or β-position. Optionally, when R1 is ¹⁸F or ¹⁹F, R2 is ¹⁸F or ¹⁹F in α- or β-position. Optionally, when R1 is OH, R2 is ¹⁸F or ¹⁹F in α- or β-position. Optionally, when R1 is CH₃, R2 is ¹⁸F or ¹⁹F in α- or β-position. Optionally, when X is O, Y is NH. Optionally, when X is NH, Y is O. Optionally, when X is NH, Y is NH. Optionally, when X is NH, Y is CH₂. Optionally, when X is O, Y is S. Optionally, when X is NH, Y is S.

In another embodiment, the present invention provides compounds 1, 2, 3, 4, and 5 of the following formulas 1-5:

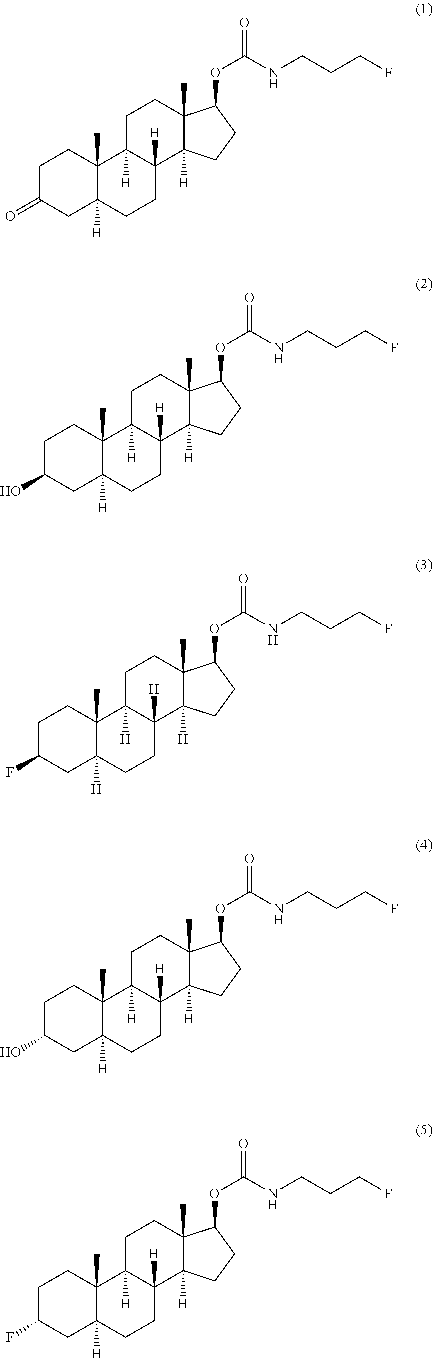

The compound (1) is also referred to herein as RB390. The compound (2) is also referred to herein as 3b-hydroxy-RB448. The compound (3) is also referred to herein as 3b-fluoro-RB448. The compound (4) is also referred to herein as 3a-hydroxy-RB448. The compound (5) is also referred to herein as 3a-fluoro-RB448.

Given the differential binding capacity and favorable radioactivity pattern, formula I, compounds 1, 2, 3, 4, and 5 of the present invention represent novel imaging ligands with diagnostic potential for PC.

In another embodiment, the present invention relates to a radiopharmaceutical composition comprising the compound according to formula I, optionally, compounds 1, 2, 3, 4, or 5 together with a biocompatible carrier in a form suitable for administration to a mammal. A biocompatible carrier can be any carrier known in art. The compound is adminstered by any known suitable method in art, optionally, by intravenous injection.

In another aspect, the present invention provides a method of diagnosing and monitoring prostate cancer and its metastases in a subject. The method comprises contacting the compound of formula I, optionally, compounds 1, 2, 3, 4, or 5 to the subject, and imaging the subject by means of positron emission tomography, wherein the compound is employed as a tracer. Contacting the compound of formula I, compounds 1, 2, 3, 4, or 5 to the subject is performed, optionally, by intravenous injection by any known method in art.

In another embodiment, the present invention relates to use of the compound according to formula I, optionally, compounds 1, 2, 3, 4, or 5 in diagnosing and monitoring prostate cancer and metastases.

### Materials and Methods

### Material and Tissue

¹⁸F-fluoride was produced at Swan Isotopen AG (Berne, Switzerland). Anhydrous solvents for reactions were obtained by filtration through activated Al₂O₃. Column chromatography (CC) was performed on silica gel (Fluka, Buchs, Switzerland, average particle size: 51 μm). Thin layer chromatography (TLC) was performed on silica gel plates (Machery-Nagel, Oensingen, Switzerland, 0.25 mm, UV254). Visualization was performed by staining in dip solution [Cer(IV)-sulfate (10.5 g), phosphormolybdenic acid (21 g), conc. H₂SO₄ (60 ml), H₂O (900 ml)] followed by heating with a heat gun. Radio-TLC plates were analyzed using a Cyclone Storage Phosphor Scanner (Perkin Elmer). Radioactivity was determined with a dose calibrator. The radiochemical yield is decay-corrected to the beginning of synthesis time. Nuclear magnetic resonance (NMR) spectra were recorded on a Bruker Avance 300 or a Bruker Avance II 400 spectrometer at 300 MHz (¹H NMR) or 100 MHz (¹³C NMR) or 376 MHz (¹⁹F NMR) in CDCl₃. δ in ppm relative to residual undeuterated solvent [CHCl₃: 7.24 ppm (¹H) and 77.0 ppm (¹³C)]. Nanospray ionization (NSI⁺, positive mode) high resolution mass spectra (HRMS) were recorded on an Applied Biosystem Sciex QSTAR Pulsar instrument.

Lysate was prepared from healthy mouse liver and from a resected unaffected part of a human liver at time a tumor was removed. Tissue collected in ice-cold culture medium was immediately processed.

### Synthesis of RB390 (Compound 1)

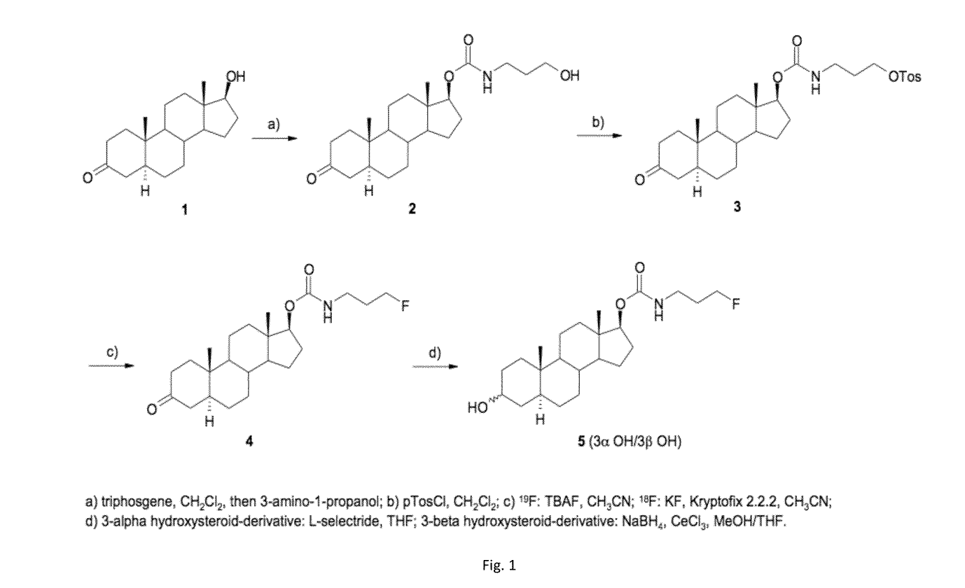

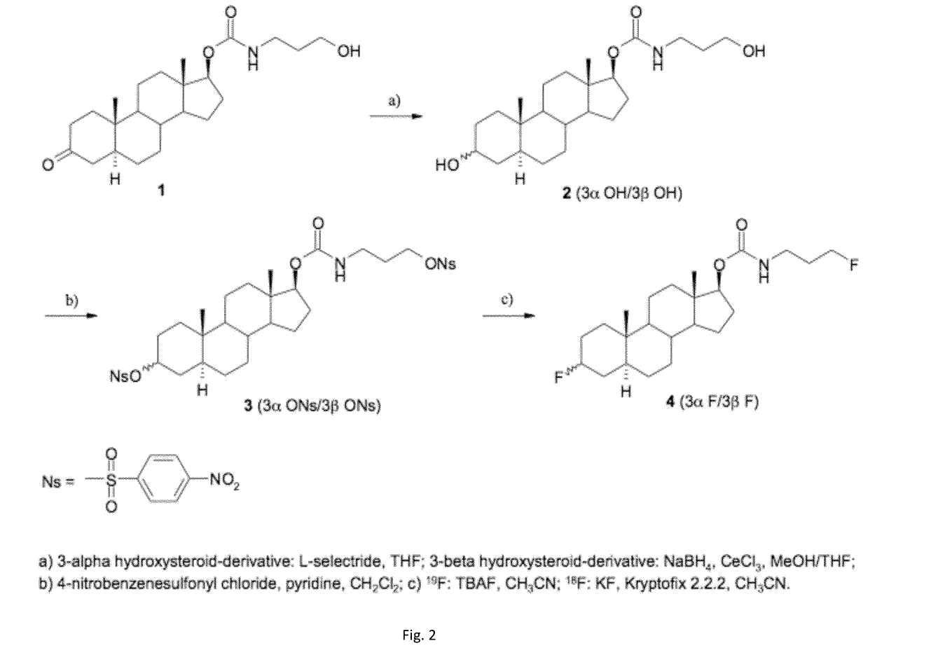

The compounds of formula I and accordingly compounds of 1, 2, 3, 4, and 5 of the present invention may be prepared by the method such as illustrated in or analogous to the method herein. Additional details are illustrated in FIG. 1 and FIG. 2. Tetrabutylammonium fluoride (186 mg, 0.59 mmol) was added to a solution of compound 3 (FIG. 1) (214 mg, 0.39 mmol) in dry CH₃CN (6 ml). The mixture was stirred for 30 min at 80° C. After evaporation of the solvent the crude material was purified by CC (hexane/EtOAc 3:2) to give RB390 (109 mg, 71%) as white foam. TLC (hexane/EtOAc 4:3) R_{f}=0.32; ¹H NMR (300 MHz, CDCl₃) δ: 0.68-2.41 (m, 24H), 0.75 (s, 3H, CH₃), 0.98 (s, 3H, CH₃), 3.29 (q, 2H, J=6.3 Hz, 12.7 Hz, N—CH₂), 4.50 (dt, 2H, J_{HF}=47.2, J_{HH}=5.7 Hz, CH₂—F), 4.50 (t, 1H, J=8.4 Hz, 17α-H), 4.81 (br s, 1H, NH); ¹³C NMR (100 MHz, CDCl₃) δ: 11.35, 12.01, 20.82, 23.33, 27.60, 28.67, 31.09, 35.09, 35.60, 36.76, 37.56, 37.99, 38.38, 42.48, 44.54, 46.50, 50.38, 53.62, 82.92, 83.15, 156.68, 211.79; ¹⁹F NMR (376 MHz, CDCl₃) δ: −220.87 (s). NSI⁺-HRMS: calculated for C₂₃H₃₇O₃NF ([M+H]⁺) 394.2742, found 394.2752.

### Radiochemical Synthesis of ¹⁸F-RB390 (Compound 1)

¹⁸F-fluoride ion (2.39 GBq) in 2 ml H₂O was concentrated on an anion exchange column (Sep-Pak QMA light, Waters) and eluted with a solution of Kryptofix 2.2.2 (15 mg, 0.04 mmol) and K₂CO₃ (10 μl, 1.0M aq. solution) in H₂O/acetonitrile 2:45 (940 μl) into a sealable test tube with a stirring bar. Water was azeotropically evaporated at 110° C. using dry acetonitrile (3×500 μl) under a stream of nitrogen. Precursor 3 (FIG. 1) (5 mg, 9.2 mmol) in acetonitrile (500 μl) was added to react at 90° C. for 10 min. The solvent was evaporated at 90° C. under a stream of nitrogen. The crude radioligand ¹⁸F-RB390 was dissolved in hexane/EtOAc 2:1 and purified by CC (5.0 g SiO₂). ¹⁸F-RB390 was eluted from the column with hexane/EtOAc 2:1. Fractions were monitored by radio-TLC. The identity of the radiolabeled compound was confirmed by co-migration of unlabeled standard. ¹⁸F-RB390 was produced within 3 h in a decay-corrected radiochemical yield of 36-57% (n=3) with a radiochemical purity of >95% as gauged by radio-TLC.

### Synthesis of 3b-Hydroxy-RB448 (Compound 2)

Cerium(III) chloride heptahydrate (33 mg, 0.09 mmol) was added to a solution of RB390 (32 mg, 0.08 mmol) in MeOH/THF (2:1, 1.5 ml). Then, NaBH₄ (3 mg, 0.08 mmol) was added and the mixture was stirred at RT. After completion (2 h), a saturated aqueous solution of NaHCO₃ (20 ml) was added and the mixture was extracted with EtOAc (20 ml). The extract was dried (Na₂SO₄) and evaporated in vacuum. The crude residue was purified by CC on silica gel (hexane/EtOAc 4:3) to give RB448 (20 mg, 62%) as white foam. TLC (hexane/EtOAc 4:3) R_{f}=0.27; ¹H NMR (300 MHz, CDCl₃) δ: 0.62 (td, 1H, J=11.7, 4.1 Hz), 0.72 (s, 3H, CH₃), 0.78 (s, 3H, CH₃), 0.82-2.14 (m, 24H), 3.29 (q, 2H, J=6.4 Hz, N—CH₂), 3.51-3.61 (m, 1H, CH(3)-OH), 4.49 (dt, 2H, J_{HF}=47.2, J_{HH}=5.7 Hz, CH₂—F), 4.50 (t, 1H, J=8.4 Hz, 17a-H), 4.79 (br s, 1H, NH); ¹³C NMR (100 MHz, CDCl₃) δ: 12.11, 12.31, 20.72, 23.43, 27.70, 28.56, 31.48, 31.56, 35.32, 35.55, 37.00, 37.62, 38.18, 42.59, 44.85, 50.68, 54.33, 71.23, 83.22, 156.80. NSI⁺-HRMS: calculated for C₂₃H₃₈O₃NF ([M+H]⁺) 396.2908, found 396.2898.

### Synthesis of 3b-Hydroxy-¹⁸F-RB448 (Compound 2)

Cerium(III) chloride heptahydrate is added to a solution of ¹⁸F-RB390 in MeOH/THF (2:1). Then, NaBH₄ is added and the mixture is stirred at RT. After completion the solvent is evaporated at 80° C. under a stream of nitrogen. The crude radioligand is purified by CC on silica gel (or by HPLC). Fractions are monitored by radio-TLC. The identity of the radiolabeled compound is confirmed by co-migration of unlabeled standard.

### Synthesis of 3a-Hydroxy-RB448 (Compound 4)

Lithium tri-sec-butylborohydride (1M in THF) is added to a solution of RB390 in dry THF and the mixture is stirred at RT. After completion, a saturated aqueous solution of NaHCO₃ is added and the mixture is extracted with EtOAc. The extract is dried (Na₂SO₄) and evaporated and the crude residue is purified by CC on silica gel.

### Synthesis of 3a-Hydroxy-¹⁸F-RB448 (Compound 4)

Lithium tri-sec-butylborohydride (1M in THF) is added to a solution of ¹⁸F-RB390 in dry THF and the mixture is stirred at RT. After completion, the solvent is evaporated at 80° C. under a stream of nitrogen. The crude radioligand is purified by CC on silica gel (or by HPLC). Fractions are monitored by radio-TLC. The identity of the radiolabeled compound is confirmed by co-migration of unlabeled standard.

### Synthesis of 3b-fluoro-RB448 (compound 3) and 3a-fluoro-RB448 (compound 5)

Tetrabutylammonium fluoride (3 equivalents) is added to a solution of the corresponding nosylate precursor 3 (FIG. 2) (1 equivalent) in dry CH₃CN and the mixture is stirred at 90° C. After completion, a saturated aqueous solution of NaHCO₃ is added and the mixture is extracted with EtOAc. The extract is dried (Na₂SO₄) and evaporated and the crude residue is purified by CC on silica gel.

### Radiochemical Synthesis of 3b-¹⁸F-fluoro-RB448 (compound 3) and 3a-¹⁸F-fluoro-RB448 (compound 5)

¹⁸F-fluoride ion in H₂O is trapped on an anion exchange column (Sep-Pak QMA light, Waters) and eluted with a mixture of Kryptofix 2.2.2 (15 mg, 0.04 mmol) and K₂CO₃ (100, 1.0M aq. solution) in H₂O/acetonitrile 2:45 (940 μl) into a sealable test tube with a stirring bar. Water is azeotropically evaporated at 110° C. using dry acetonitrile (3×500 μl) under a gentle stream of nitrogen. The corresponding nosylate precursor 3 (FIG. 2) (5 mg) in acetonitrile (500 μl) is added to react at 90° C. for 15 min. The solvent is evaporated at 90° C. under a stream of nitrogen. The crude radioligand is purified by CC on silica gel (or by HPLC). Fractions are monitored by radio-TLC. The identity of the radiolabeled compound is confirmed by co-migration of unlabeled standard.

### Induced Fit Docking

Schrodinger 2012 package Induced Fit Docking workflow (IFD) was used to predict the orientation, or pose, of compound RB390 relative to the WT (PDB 1137) and mutant receptors (PDB 2OZY), similar to the previous known protocols (For example, see Andrieu T, Bertolini R, Nichols S E, et al. A novel steroidal antiandrogen targeting wild type and mutant androgen receptors. Biochem Pharmacol. Dec. 1, 2011; 82(11):1651-1662; and Sherman W, Day T, Jacobson M P, Friesner R A, Farid R. Novel procedure for modeling ligand/receptor induced fit effects. J Med Chem. Jan. 26, 2006; 49(2): 534-553). IFD is a three step iterative procedure that combines Glide (Maestro v.9.3.023) rigid receptor docking with Prime protein structure prediction for adjustment of the protein conformation to the ligand (For example, Friesner R A, Banks J L, Murphy R B, et al. Glide: a new approach for rapid, accurate docking and scoring. 1. Method and assessment of docking accuracy. J Med Chem. Mar. 25, 2004; 47(7):1739-1749.). All structures were prepared for docking using the protein preparation wizard, by adding bond orders and hydrogen, and removing all waters greater than 5 Å away from the ligand binding site. The mutant crystal structure 2OZ7 was aligned to AR WT structure 1137 for comparison of ligand orientation (Bohl C E, Miller D D, Chen J, Bell C E, Dalton J T. Structural basis for accommodation of nonsteroidal ligands in the androgen receptor. J Biol Chem. Nov. 11, 2005; 280(45):37747-37754; and Sack J S, Kish K F, Wang C, et al. Crystallographic structures of the ligand-binding domains of the androgen receptor and its T877A mutant complexed with the natural agonist dihydrotestosterone. Proc Natl Acad Sci USA. Apr. 24, 2001; 98(9):4904-4909.). The ligand was docked using Glide S P, and the protein was refined with Prime. Select residues are represented as alanine at the initial docking step to provide more space. When docking RB390, residues 876, 877, and 891 were modeled as alanine, as differences in the 1137 and 2OZ7 crystal structures indicated variations in those side chains. Generated relaxed protein conformations and Glide XP were then used to redock the compound and rank the orientations.

### Qualitative Stability Measurements of RB390 and ¹⁸F-RB390 in Human Blood and Plasma

RB390 was incubated with plasma of a healthy volunteer at 37° C. for 24 h, extracted and analyzed by TLC. ¹⁸F-RB390 (3MBq/300 μl) was added to 300 μl of human blood samples, incubated over various time points at 37° C. in an Eppendorf-shaker, extracted with 100 μl of hexane/EtOAc (2:3) and analyzed as mentioned above.

### Semiquantitative Stability Measurements of ¹⁸F-RB390 in Mouse and Human Liver Lysates in Absence and Presence of Glycyrrhetinic Acid (GA)

Lysates of fresh mouse and human liver tissue were prepared using sucrose buffer (250 mM sucrose, 10 mM Tris-Base, pH7.5) with sonification. Protein concentrations were determined and samples immediately frozen at −70° C. ¹⁸F-RB390 (3MBq/300 μl) was added to lysates in absence and presence of 50 μM GA (see Pirog E C, Collins D C. Metabolism of dihydrotestosterone in human liver: importance of 3alpha-and 3beta-hydroxysteroid dehydrogenase. J Clin Endocrinol Metab. September 1999; 84(9):3217-3221 and Latif S A, Conca T J, Morris D J. The effects of the licorice derivative, glycyrrhetinic acid, on hepatic 3 alpha-and 3 beta-hydroxysteroid dehydrogenases and 5 alpha-and 5 beta-reductase pathways of metabolism of aldosterone in male rats. Steroids. February 1990; 55(2): 52-58.), extracted and spotted as described above.

Semiquantitative stability measurements of precursor 3 (FIG. 1) during an 1 h sterilization process using 120° C. (Procedure used in the hospital pharmacy of the University Hospital Berne for sterilizing solutions to be injected into humans). During this sterilization process the precursor 3 was degraded, however the alternate sterilization process using a Millipore sterile filter device with a hydrophilic PTFE membrane 0.2 μm pore size is suitable for this purpose.

### Cell Cultures

C4-2 cells (see Thalmann G N, Sikes R A, Wu T T, et al. LNCaP progression model of human prostate cancer: androgen-independence and osseous metastasis. Prostate. Jul. 1, 2000; 44(2):91-103 July 101; 144(102).) were grown in complete RPMI-1640 medium. PC-3 and receptor-free COS-7 cells were cultured in Dulbecco's modified Eagle's medium (DMEM) supplemented with 10% FBS. Cells were maintained at 37° C. in 5% CO₂ air balance. Steroids were dissolved in ethanol with 0.1% final concentration in cultures.

### Plasmids and Transfections

The following plasmids were used: pSG5-AR with full-length cDNA of human WT-AR (Goepfert C, Gazdhar A, Frey F J, Frey B M. Effect of electroporation-mediated diphtheria toxin A expression on PSA positive human prostate xenograft tumors in SCID mice. Prostate. Jun. 1, 2011; 71(8):872-880.), progesterone receptor (pSG5-PR-alpha, pSG5-pPRbeta, PR), glucocorticoid (pCMV5-hGR-alpha, GR) and mineralocorticoid receptors (pRShMR, MR), estrogen receptors (pCMV5-ER-alpha, pCMV5-ER-beta, ER). T877A-AR was generated by site-directed mutagenesis as described in Andrieu T, Bertolini R, Nichols S E, et al. A novel steroidal antiandrogen targeting wild type and mutant androgen receptors. Biochem Pharmacol. Dec. 1, 2011; 82(11):1651-1662. Cells seeded on 6-well plates (density: 5×10⁵/well) were transfected 96 h later with 1 μg of empty (pcDNA3) or human AR encoding plasmids using 3 μl of FuGENE-HD transfection reagent (Roche, Rotkreuz, Switzerland). One day after transfection the medium was changed to 2% charcoal treated (CT) FBS containing medium.

### Competitive Binding Assay

In 24 wells 50′000 COS-7 cells were seeded and transfected with 500 ng of receptor encoding plasmids. One day later, cells were washed and cultured in 0.5 ml of phenol-red free DMEM with 2% CT-FBS for another 24 h. RB390 or RB448 was added together with the radioligand [1,2,4,5,6-³H]-DHT (112.9 Ci/mmol, Amersham, Otelfingen, Switzerland) and incubated together with the cells for 1 h at 37° C., 5% CO₂. Supernatant and cells were mixed with scintillation liquid (Irgasafe, Perkin Elmer) and radioactivity counted (Tri-Carb 2000CA, Canberra Packard). The percentage of binding in presence and absence of derivative was calculated relatively to total dpm and results expressed as percentage of binding relative to the ³H-tracer alone (100%).

### Ligand Uptake by Transfected Cells

Forty-eight hours after transfection medium was changed to DMEM, 0% FBS, cells incubated with 0.1-100 kBq of ¹⁸F-FDG, ¹⁸F-FCH or ¹⁸F-RB390 for 10 min and washed 3×10 min with DMEM, 2% FBS at 37° C. Then cells were washed with ice cold PBS. Collected medium and PBS were considered as the unbound fraction. Cells were incubated for 5 min in glycine buffer 50 mM (pH 2.8), washed with PBS (membrane fraction) and lysed with RIPA buffer (Sigma, R0278) (cellular fraction). The radioactivity was measured by gamma counting. Cellular uptake of radioactivity was corrected by the protein amount (BCA assay, Pierce). For specific uptake, cells were incubated with ¹⁸F-RB390 in presence or absence of 10⁻⁶M unlabeled DHT added during the 10 min incubation and 3 washing steps.

### Xenografts

Male SCID-mice (Charles River, Sulzfeld, Germany) were subcutaneously injected with C4-2 and PC-3 cells into both shoulders as previously described (21).

### Biodistribution and PET-Imaging

Biodistribution studies and PET-Imaging were performed in C4-2 and PC-3 tumor-bearing SCID-mice by injecting tracers of 1MBq/0.1 ml of ¹⁸F-FDG or ¹⁸F-FCH or ¹⁸F-RB390 into the tail vein. After 1 h, mice were scanned on a combined PET/CT scanner (128-slice mCT, Siemens, Knoxville) with 200 mAs, 100KeV and a pitch of 0.5. Images were reconstructed at 0.6 mm thickness using a H31s medium smooth plus kernel. The PET images were acquired over 15 min and iteratively reconstructed (4 iterations, 12 subsets) using a Gaussian filter. For biodistribution studies mice were sacrificed using CO₂ after 1-3 h, designated organs and tissues harvested and weighed. Radioactivity was measured in a Gamma-counter (Wizard, Perkin Elmer) and the percentage of injected dose per gram of tissue (% ID/g) calculated.

### Statistical Analysis

Calculations were performed using GraphPad Prism 5.0 software (GraphPad Software, Inc.) Values are given as mean±SEM. For statistical analysis of biodistribution studies the one-way ANOVA test was used. Results were considered significant when p<0.05.

### Results

### Synthesis and Radiochemistry

Compound RB390 was synthesized in a 3-step procedure starting with 5αDHT (FIG. 1). The radiochemical synthesis of ¹⁸F-RB390 was accomplished according to a standard procedure: ¹⁸F-fluoride ion was activated by addition of K₂CO₃ and Kryptofix 2.2.2 and incorporated into precursor 3 in high radiochemical yields of 36-57% (decay-corrected, n=3). The resulting radiolabeled product was purified by column chromatography. A single not identified radioactive by-product could be readily separated from ¹⁸F-RB390. The identity of the product was confirmed by co-migration on TLC with unlabeled RB390. There was no evidence of chemical impurities in the collected fractions and the radiochemical purity was >95% as gauged by radio-TLC. Radiochemical synthesis and purification were accomplished within 3 h. The identity of the metabolite of RB390/¹⁸F-RB390 was elucidated by co-migration with 3-hydroxysteroid RB448.

### Biological Stability Experiments

The stability of RB390 and ¹⁸F-RB390 was assessed by incubating them in human plasma and blood for 0-24 h or mouse/human liver homogenate for 0-3 h in presence and absence of glycyrrhetinic acid (GA). Excellent stability was observed in human blood over 90 min, while a small percentage was metabolized thereafter. According to radio-TLC the metabolite was more polar than ¹⁸F-RB390. Using NaBH₄, reduction of the 3-keto group of RB390 yielded the corresponding 3-hydroxysteroid (3-hydroxy-RB448) co-migrating on TLC with the radiolabeled metabolite. A similar stability in plasma was observed. Since steroid metabolism occurs mainly in the liver, ¹⁸F-RB390 was incubated in human and murine liver homogenate. Equivalent protein concentrations of both homogenates were used. After 60 min metabolite 3-hydroxy-¹⁸F-RB448 was detected in murine samples, while 3-hydroxy-¹⁸F-RB448 was found in human liver homogenate after 20 min. In order to analyze whether the enzymatic activity could be inhibited by GA, 5004 of GA was added to human and murine liver homogenate. The degradation however was not prevented. Consequently similar binding studies for 3-hydroxy-RB448 as for RB390 were performed.

### Competitive Binding Assay

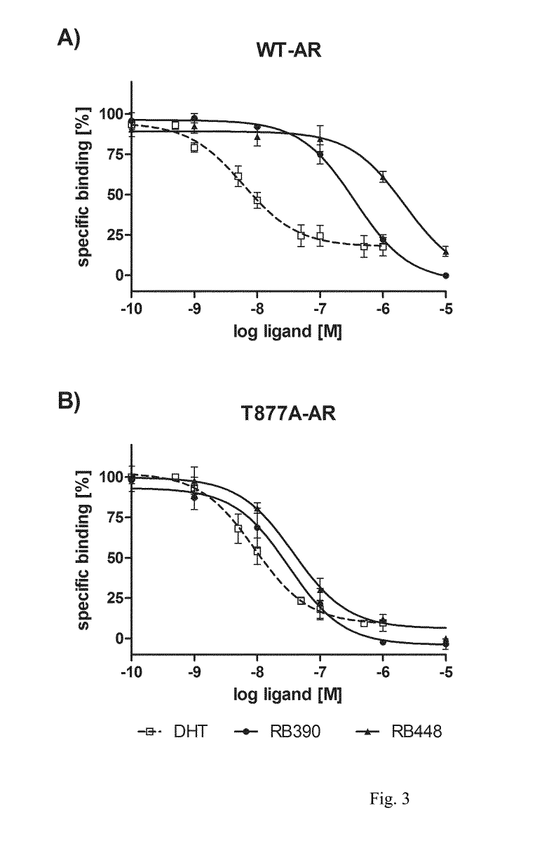

Competitive binding studies for RB390 and 3-hydroxy-RB448 to bind to WT-AR or T877A-AR and displacing ³H-DHT were performed in COS-7 cells transiently transfected with the corresponding plasmids (FIG. 3). Both, RB390 and 3-hydroxy-RB448, inhibited the binding of ³H-DHT to WT-AR or T877A-AR dose-dependently (FIG. 3A, B). The IC₅₀ values of RB390 were 357 nM for WT-AR and 32 nM for T877A-AR, while values of RB448 were 2205 nM for WT-AR and 37.7 nM for T877A-AR (FIG. 3A, B). Thus there is a higher affinity of both ligands for T877A-AR. By comparison, the IC₅₀ value for DHT was 6 nM for WT-AR and 9 nM for T877A-AR. The binding capacity of both ligands to other receptors (n=2) was very low, except for ERb and MR (around 20% RBA with respect to ³H-aldosterone or ³H-estradiol=100%).

### Modeling

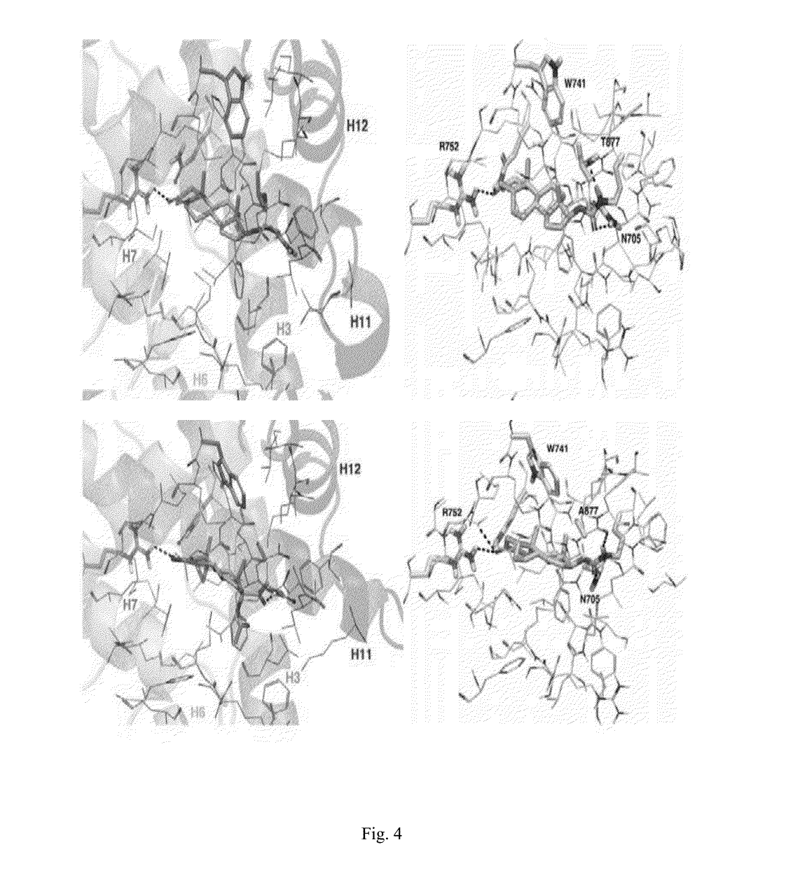

To predict how RB390 interacts with the WT-AR and T877A-AR binding domain, we performed docking studies with Schrodinger's Induced Fit Docking workflow. It has previously shown that IFD can reproduce binding modes of known androgens and antagonists (Andrieu T, Bertolini R, Nichols S E, et al. A novel steroidal antiandrogen targeting wild type and mutant androgen receptors. Biochem Pharmacol. Dec. 1, 2011; 82(11):1651-1662). RB390 was docked into the WT-AR and T877A-AR. The best-docked predictions were aligned and compared to co-crystallized ligands (FIG. 4). Results indicate a similar binding orientation for both the WT and mutant T877A-AR. Crystallographic and predicted poses reveal steroidal core for DHT and for DHT derivatives, as well as side chains that point towards the N-terminal end of helix H12, all represent similar orientations.

### Uptake of ¹⁸F-RB390 by Transfected COS-7 Cells

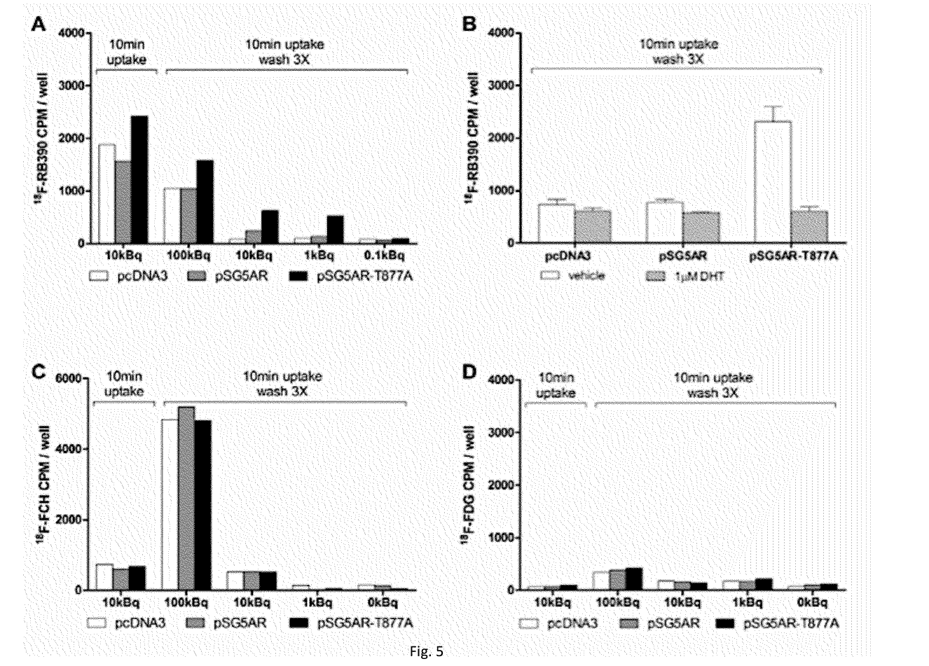

Differential uptake of ¹⁸F-RB390 by COS-7 cells transfected with pcDNA3, pSG5AR, and pSG5AR-T877A is presented in FIG. 5. After incubation of 10 kBq of ¹⁸F-RB390 for 10 min, the mean radioactivity in pSG5AR-T877A transfected cells was in the range of 13.3 cpm/μg of protein, whereas a lower amount of 10.3 and 8.6 cpm/μg was measured in pcDNA3 and pSG5-AR transfected control cells (FIG. 5A). Additional washing steps significantly reduced (up to 25 times) unspecific uptake into transfected cells and uptake of ¹⁸F-RB390 in pSG5AR-T877A transfected cells increased. Tracer uptake of pSG5AR-T877A transfected cells was 2.5 times higher compared to pSG5AR-transfected cells when consequent washes were performed, but only 1.5 times increase was detected without washing. Reducing the amount of radioligand additionally increased selectivity towards the T877A-AR variant (3.7 fold higher, 1 kBq), while increased quantity of radioligand reduced selectivity (1.5 fold, 100 kBq). Uptake of radioligand was minimal when cells were incubated with ¹⁸F-RB390 in presence of excessive unlabeled DHT, demonstrating that the uptake was AR specific (FIG. 5B). Prolongation of the incubation period up to 1 h did not increase the amount of radioactivity within the cells indicating that the equilibrium was reached within minutes. Incubation of transfected cells with ¹⁸F-FDG and ¹⁸F-FCH did not reveal any preferential uptake (FIG. 5C, D) concluding that the cellular metabolism was identical for both conditions.

### Animal Biodistribution Studies

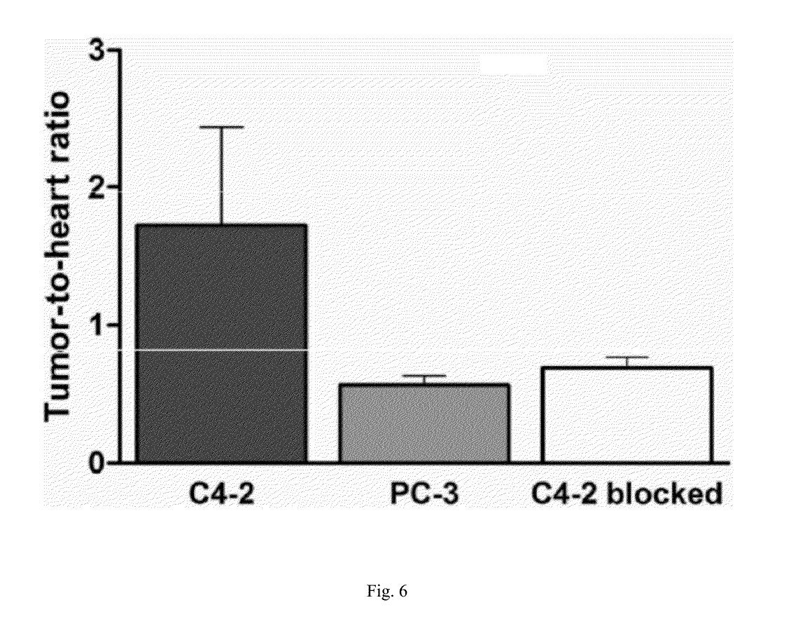

Biodistribution data of the tracers ¹⁸F-FDG, ¹⁸F-FCH and ¹⁸F-RB390 in C4-2 and PC-3 tumor bearing SCID-mice are summarized in Table 1. ¹⁸F-FDG and ¹⁸F-FCH were injected and mice sacrificed 1 h after tracer injection as previously reported (Smith G, Zhao Y, Leyton J, et al. Radiosynthesis and pre-clinical evaluation of [(18)F]fluoro[1,2-(2)H(4)]choline. Nucl Med Biol. January 2011; 38(1):39-51 and DeGrado T R, Reiman R E, Price D T, Wang S, Coleman R E. Pharmacokinetics and radiation dosimetry of 18F-fluorocholine. Journal of Nuclear Medicine. January 2002; 43(1):92-96.). All radiotracers displayed a high uptake in blood rich organs (heart, spleen) 1 h after application. ¹⁸F-FDG and ¹⁸F-FCH were excreted predominantly by the kidney, while ¹⁸F-RB390 was metabolized by the liver and eliminated mainly via the intestine. Uptake of ¹⁸F-RB390 by the brain was nine times lower than for ¹⁸F-FCH and fifty-three times lower than for ¹⁸F-FDG (p<0.0001). ¹⁸F-RB390 showed a high uptake in T877A-AR positive C4-2 tumors compared to receptor negative PC-3 tumors. Specificity was verified by excessive intravenous unlabelled DHT injection leading to a strongly reduced incorporation within this group (Tab. 1, FIG. 6). One hour after injection the tumor-to-heart ratios of C4-2 tumors were 0.4:1 for ¹⁸F-FCH and 0.2:1 for ¹⁸F-FDG, whereas the ratio of ¹⁸F-RB390 was 1.2:1. In PC-3 control tumors the ratio of ¹⁸F-RB390 was 0.9:1 similar to the relatively high tracer blood level at this time. After 3 h the ¹⁸F-RB390 tumor-to-heart ratios were highest in C4-2 tumors (1.9:1) and lower in control tumors (0.5:1) (FIG. 6).

### PET Imaging

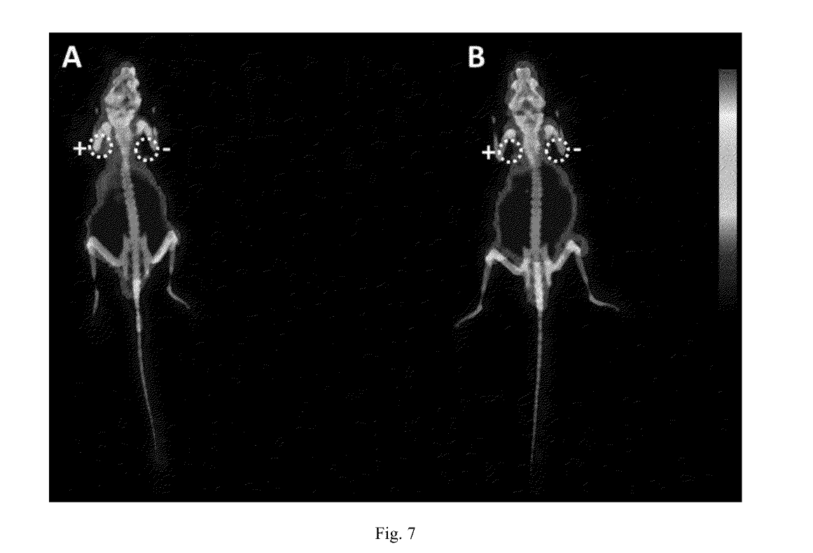

FIG. 7 demonstrates the in vivo uptake of ¹⁸F-RB390 by T877A-AR positive C4-2 tumors at 3 h post injection. In comparison, no tumor signal was detected in the PC-3 AR-negative xenografts. Specificity to mutated T877A-AR was demonstrated by injecting nonradioactive DHT. The resulting PET scan clearly showed a decrease in tumor uptake 3 h after injecting the imaging probe and DHT.

### Further Results and Advantages of Compounds of Present Invention

PC is a serious public health problem associated with significant emotional, practical and financial expenses. Moreover there is a need to find better ways to distinguish between patients with PCs who show poor prognosis from those who do better. New therapeutic approaches are under development using specific endpoints with the general goal to control, reduce or yet eliminate disease manifestations (e.g., prostate-specific antigen or imaging findings) and to impede or prevent future disease appearance. At present, imaging plays a precious diagnostic role in many aspects of this disease. Thus the central requirements for clinical applications of radiotracers for cancer diagnosis are not only excellent specificity, but also suitable biodistribution properties with a maximal tumor targeting and minimal background situation. In the past various scientists have designed non-steroidal and steroidal AR radioligands, but the majority are steroidal analogs (see for example, Beattie B J, Smith-Jones P M, Jhanwar Y S, et al. Pharmacokinetic assessment of the uptake of 16beta-¹⁸F-fluoro-5alpha-dihydrotestosterone (FDHT) in prostate tumors as measured by PET. J Nucl Med. February 2010; 51(2):183-192; Parent E E, Carlson K E, Katzenellenbogen J A. Synthesis of 7alpha-(fluoromethyl)dihydrotestosterone and 7alpha (fluoromethyl) nortestosterone, structurally paired androgens designed to probe the role of sex hormone binding globulin in imaging androgen receptors in prostate tumors by positron emission tomography. J Org Chem. Jul. 20, 2007; 72(15):5546-5554; and Parent E E, Dence C S, Sharp T L, Welch M J, Katzenellenbogen J A. 7alpha-¹⁸F fluoromethyldihydrotestosterone and 7alpha-18F-fluoromethyl-nortestosterone: ligands to determine the role of sex hormone-binding globulin for steroidal radiopharmaceuticals. J Nucl Med. June 2008; 49(6):987-994.). Since the major circulating androgen, testosterone, has lower affinity for the AR than its metabolite DHT, most scientists placed their focus on DHT. For radioligand production using short-lived isotopes, viable strategies incorporate the radioisotope near or at the end of the synthetic scheme (Parent E E, Carlson K E, Katzenellenbogen J A. Synthesis of 7alpha-(fluoromethyl)dihydrotestosterone and 7alpha-(fluoromethyl)nortestosterone, structurally paired androgens designed to probe the role of sex hormone binding globulin in imaging androgen receptors in prostate tumors by positron emission tomography. J Org Chem. Jul. 20, 2007; 72(15):5546-5554.). The radio-fluorination and purification of DHT-derivative ¹⁸F-RB390 was accomplished within 3 h with a purity of more than 95% and a yield of 36-57%. In addition, investigators discussed several limitations of the various radioligands proposed, such as instability, defluorination, metabolism, unfavorable binding to sex hormone binding protein or binding in a promiscuous manner to other nuclear receptors (Mankoff D A, Link J M, Linden H M, Sundararajan L, Krohn K A. Tumor receptor imaging. J Nucl Med. June 2008; 49 Suppl 2:1495-1635; and see Liu A, Carlson K E, Katzenellenbogen J A. Synthesis of high affinity fluorine-substituted ligands for the androgen receptor. Potential agents for imaging prostatic cancer by positron emission tomography. J Med Chem. May 29, 1992; 35(11):2113-2129.). ¹⁸F-RB390 of the present invention, however has the enormous advantage that mainly one metabolite was formed within 180 min, when incubated in human or mouse liver homogenate at 37′C and the binding to other nuclear receptors was rather marginal. This type of degradation could however not be inhibited by GA, indicating that 3α-HSD, an enzyme insensitive to GA, and not 3β-HSD, sensitive to GA was involved (Latif S A, Conca T J, Morris D J. The effects of the licorice derivative, glycyrrhetinic acid, on hepatic 3 alpha-and 3 beta-hydroxysteroid dehydrogenases and 5 alpha-and 5 beta-reductase pathways of metabolism of aldosterone in male rats. Steroids. February 1990; 55(2):52-58.). The metabolism for RB390 to 3-hydroxy-RB448 however is not crucial because both DHT derivatives have similar binding properties (FIG. 3) with preference to the mutation T877A. In this respect ¹⁸F-RB390 represents an excellent, unique candidate for PET imaging.

In one embodiment, the present invention designs and develops a specific detection system for targeting T877A-AR or similar AR mutations located in the ligand binding domain (LBD), since mutated ARs are present only in PC (see for example, Sun C, Shi Y, Xu L L, et al. Androgen receptor mutation (T877A) promotes prostate cancer cell growth and cell survival. Oncogene. Jun. 29, 2006; 25(28):3905-3913; and see Moehren U, Papaioannou M, Reeb C A, et al. Wild-type but not mutant androgen receptor inhibits expression of the hTERT telomerase subunit: a novel role of AR mutation for prostate cancer development. FASEB J. April 2008; 22(4): 1258-1267.).

On the basis of in vitro binding studies using COS-7 cells transfected with either WT-AR or T877A-AR mutant plasmids RB390 was selected from a variety of candidates presenting a favorable IC₅₀ value for the T877A-AR (FIG. 3). As mentioned, the stability of ¹⁸F-RB390 assessed in human blood and human and mouse liver homogenate revealed an excellent result in blood. The metabolite 3-hydroxy-RB448 demonstrated an even more favorable selectivity to mutant T877A-AR as the parent compound RB390, indicating, that the 3rd position in the A-ring might be a relevant discriminator for binding. The compounds of the present invention target preferentially T877A-AR, only present in PC cells, which was reached by performing binding experiments with either RB390 or its metabolite 3-hydroxy-RB448. Cell cultures exposed to various doses of radioligand ¹⁸F-RB390 presented evidence that lower doses (1 and 10 kBq) of RB390 discriminated, in a more selective manner, between WT-AR versus T877A-AR compared to ¹⁸F-FCH or ¹⁸F-FDG. The washing procedures improved the selectivity even more when ¹⁸F-RB390 was considered (FIG. 5A, B), while the radioactivity was eliminated when ¹⁸F-FCH or ¹⁸F-FDG was used. This experimental design demonstrates that the steroidal radioligand was bound with preference to T877A-AR. This statement was substantiated by the results obtained after competing the binding with unlabeled DHT in vitro. A similar effect was observed in vivo in tumor bearing SCID-mice when blocking experiments were performed.

These PET imaging studies show that the DHT-derivative RB390 was bound specifically to C4-2 tumors, whereas tracer uptake in AR-negative PC-3 tumors remained at background levels. This comparison with the AR-negative control tumor supports the specific radioligand binding as the major mechanism of localization. The convincing tumor-to-heart uptake ratios in AR-positive tumors versus AR-negative tumors (PC-3) and the resultant quality of the images improved when the imaging time was extended from 1 to 3 h after injection. The specificity of the radiolabeled DHT-derivative RB390 was proven by the blocking experiments, in which a nearly full receptor blockade could be reached with non-radiolabeled DHT. Given the differential binding capacity and favorable radioactivity pattern, the compounds of the present invention, in particular, ¹⁸F-RB390 and 3-hydroxy-¹⁸F-RB448 represents a novel, unique imaging ligand with excellent diagnostic potential for PC.

### Other Embodiments

The detailed description set forth above is provided to aid those skilled in the art in practicing the present invention. However, the invention described and claimed herein is not to be limited in scope by the specific embodiments herein disclosed because these embodiments are intended as illustration of several aspects of the invention. The embodiments set forth above can be performed and combined with other disclosed embodiments according to the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims. All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

TABLE 1
Biodistribution of ¹⁸F-FDG, ¹⁸F-FCH and ¹⁸F-RB390 in PC-3 and C4-2 Tumor-Bearing Mice

| | ¹⁸F-FCH | ¹⁸F-FDG | ¹⁸F-RB390 | | |
|---|---|---|---|---|---|
| | 1 h | 1 h | 1 h | 3 h | 3 h blocked |
| Organ (% ID/g) | (n = 5) | (n = 4) | (n = 10) | (n = 4) | (n = 3) |
| C4-2 Tumor | 31.62 ± 3.88 | 36.14 ± 8.49 | 5.28 ± 2.79 | 2.64 ± 1.90 | 1.29 ± 0.06 |
| PC-3 Tumor | 5.65 ± 1.37 | 20.78 ± 8.28 | 3.79 ± 1.35 | 0.88 ± 0.18 | 1.18 ± 0.33 |
| Testis | 13.58 ± 2.64 | 65.97 ± 12.93 | 11.96 ± 4.17 | 5.09 ± 0.61 | 5.65 ± 0.72 |
| Prostate | 48.26 ± 19.35 | 227.57 ± 126.41 | 22.96 ± 25.65 | 10.62 ± 3.37 | 29.21 ± 12.78 |
| Brain | 9.61 ± 2.13 | 56.51 ± 15.03 | 1.06 ± 0.35 | 0.46 ± 0.01 | 0.50 ± 0.02 |
| Heart | 76.93 ± 5.18 | 171.45 ± 61.48 | 4.39 ± 1.97 | 1.28 ± 0.29 | 1.63 ± 0.24 |
| Lung | 97.42 ± 23.21 | 24.14 ± 5.01 | 5.17 ± 2.06 | 1.63 ± 0.62 | 1.57 ± 0.14 |
| Liver | 48.82 ± 7.55 | 11.13 ± 3.34 | 30.21 ± 15.08 | 3.03 ± 0.52 | 5.17 ± 1.35 |
| Spleen | 66.48 ± 19.45 | 17.66 ± 6.52 | 6.99 ± 3.15 | 4.12 ± 1.61 | 5.91 ± 0.97 |
| Intestine | 22.97 ± 4.67 | 14.57 ± 1.68 | 29.64 ± 14.90 | 16.16 ± 6.78 | 7.66 ± 3.34 |
| Kidney | 164.72 ± 33.57 | 16.05 ± 1.82 | 7.93 ± 3.50 | 2.18 ± 0.68 | 1.95 ± 0.22 |
| Muscle | 10.36 ± 2.58 | 29.08 ± 10.07 | 3.25 ± 1.30 | 2.55 ± 0.46 | 2.65 ± 0.51 |
| Bone | 27.99 ± 5.95 | 38.89 ± 7.72 | 15.73 ± 6.63 | 16.57 ± 4.46 | 21.34 ± 1.46 |
| Blood | NA | NA | 6.72 ± 2.46 | 11.54 ± 3.59 | 5.46 ± 0.94 |
| Urine | NA | 1071.94 ± 275.25 | 277.25 ± 164.26 | 25.18 ± 14.45 | 64.49 ± 9.55 |

NA = not assessed.
Mean ± SEM are given.
*blocked indicates parallel injection of 3 μg nonradioactive Dihydrotestosterone via tail vein

1. A compound of the following formula I: 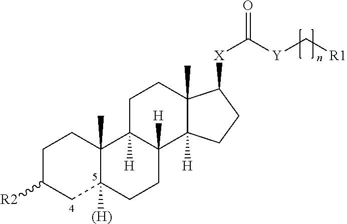
wherein R1 is ¹⁸F, ¹⁹F, OH, or CH₃; R2 is ═O, OH, ¹⁸F, or ¹⁹F; X is O or NH; Y is NH, O, CH₂, or S; n is an integer from 2-12; and 4,5-bond is a single or double bond, and wherein if R1 is CH₃, then n is an integer from 0-11.

2. The compound according to claim 1, wherein the compound is selected from the group consisting of following compounds 1, 2, 3, 4, and 5:

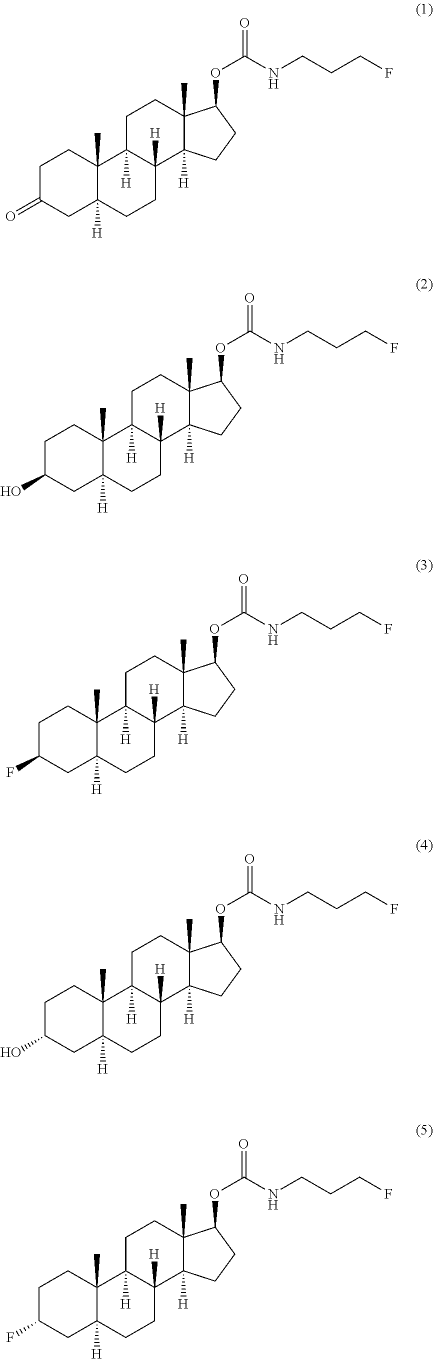

3. A radiopharmaceutical composition comprising the compound according to claim 1 together with a biocompatible carrier in a form suitable for administration to a mammal.

4. A method of diagnosing and monitoring prostate cancer in a subject, comprising contacting the compound of claim 1 to the subject, and imaging the subject by means of positron emission tomography, wherein the compound is employed as a tracer.

5. Use of the compound according to claim 1 in diagnosing and monitoring prostate cancer.
